(19) United States
(12) Patent Application Publication
(10) Pub. No.: US 2009/0005704 A1
(43) Pub. Date: Jan. 1, 2009

(54) Specimen Collection Container

(75) Inventors: Gadi Porat, Jerusalem (IL); Abraham Mates, Givat Shmuel (IL); Joel Stern, Herzliya (IL); Yehuda Cohen, Jerusalem (IL)

Correspondence Address: KREMBLAS, FOSTER, PHILLIPS & POLLICK, 7632 SLATE RIDGE BOULEVARD, REYNOLDSBURG, OH, 43068, US

(73) Assignee: ASSOCIATION FOR PUBLIC HEALTH SERVICES, Jerusalem (IL)

(21) Appl. No.: 12/097,947

(22) PCT Filed: Dec. 18, 2006
(86) PCT No.: PCT/IL06/01454

§ 371 Date: Aug. 1, 2008

(30) Foreign Application Priority Data
Dec. 22, 2005 (US) 60752400

(51) Int. Cl. A61B 5/20

(52) U.S. Cl. 600/573

(57) ABSTRACT

A liquid specimen collection container is described that includes a cup which has a base, an edge portion, and a sidewall extending from the base to the edge portion. The container additionally includes a lid disposed on the edge portion. The lid has a recessed portion which forms a protrusion extending downward into the cup. The container also includes a flexible extraction tube permanently affixed to the bottom of the protrusion with the tube reaching the base of the cup. The container further typically includes a transfer means positioned inside the recessed portion of the lid, the transfer means in liquid communication with the flexible tube. The transfer means assists in transferring liquid from the container to another vessel. A system is also described that includes the above specimen collection container and a vessel into which collected liquid is transferred from the container.

### FIELD OF THE INVENTION

The present invention relates to a specimen collection container for collecting biological fluids, particularly urine specimens.

### BACKGROUND OF THE INVENTION

Laboratory analysis is often required of biological fluids. This is particularly true of urine. Collecting and analyzing urine is problematic because collection is typically done in open top cups. After collection of the urine, the cup is closed with a tight fitting lid attached by threads or snapped on employing flange-like elements. However, for analysis purposes, smaller portions of the liquid than the amount generally collected are required; these smaller portions are typically removed from the cup into 5-10 cc evacuated test tubes.

Removal of these smaller portions is typically effected by assemblies that include an evacuated test tube and a needle cannula. One end of the needle cannula is positioned to be in liquid communication with the urine in the cup, while the second end of the needle cannula is configured to be pushed through a vacuum seal into an evacuated test tube. The resultant pressure differential generates a flow of the bodily fluid through the needle cannula into the test tube.

Evacuated test tubes may be used for collecting urine when the amount of urine collected is sufficient for the needle cannula to extend into the cup and reach the liquid. However, when only small amounts of urine are collected from a patient, the level of the liquid may not reach the cannula. In such cases, medical personnel often must tilt the specimen cup so that the maximum amount of liquid can reach the cannula while an amount of liquid remains in the cup. Even more problematically, medical personnel may try to exert additional pressure. They may try to push the test tube and needle cannula deeper into the cup. In some cases, this approach may be successful as the lid through which the cannula extends is usually formed of relatively thin plastic. However, often this approach is unsuccessful and may even be dangerous.

Pushing sharp and fragile objects, particularly when possibly biologically hazardous materials are involved, is not a good strategy. It would therefore be advantageous to develop a specimen collection container which allows for drawing off samples of sufficient size when the total amount of liquid collected is smaller than the amount for the required one or two test tubes.

When an insufficient amount of urine is obtained from a patient, the nurse or doctor will ordinarily request the patient to go to the facilities and try to produce more urine. Often, the patient is unsuccessful. He repeats this procedure several times walking back to the facilities without producing the desired result. He may then sit opposite the nurse disturbing her in various ways, such as by asking her questions. He will make visits to the cafeteria after which he will return again to disturb the nurse and in general be disruptive. The nurse therefore wants a simple quick method and device to reduce the frequency of occurrence of such situations. Therefore, it again would be advantageous for the develop of a specimen collection container which allows for drawing off samples of sufficient size when the total amount of liquid collected is small.

A typical prior art liquid specimen collection container is described in U.S. Pat. No. 6,921,395 to Carano et al. The system described therein indicates many of the elements appearing in currently constructed specimen collection containers.

### SUMMARY OF THE PRESENT INVENTION

It is an object of the present invention to provide a specimen collection container for transferring the maximum amount of collected biological fluid where the total amount of collected fluid may be small. Typically, but without intending to be limiting, the fluid is urine.

It is a further object of the present invention to provide a specimen collection container having a flexible extraction tube reaching the bottom surface of the container. Such a container does not require tilting or additional pressure to remove a portion of the collected liquid for laboratory analysis.

It is yet a further object of the present invention to provide a system for collecting a biological fluid and extracting a portion of the collected fluid for use in laboratory analysis.

There is thus provided in accordance with one aspect of the present invention a liquid specimen collection container. The container includes a cup and a lid. The cup has a base, a sidewall extending substantially transversely from the base, terminating in an edge portion, such that the base and the sidewall form a cavity. The lid is formed for leak-resistant seating on the edge portion. The lid has a recessed portion having formed therein a generally hollow protrusion terminating in a free end which, when the lid sits on the edge portion of the cup, extends into the interior of the cup. The free end is spaced apart from the base. A flexible extraction tube is present, the tube having a first end affixed to the free end of the protrusion and a second end arranged to lie on the base of the cup. The second end of the flexible extraction tube is immersed in a liquid disposed in the cup. When a suction force is applied to the recessed portion of the lid, the hollow protrusion and the flexible extraction tube are operative to transfer the suction force to the liquid, causing liquid to be drawn into the tube and through the protrusion and out of the collection container.

In a further embodiment of the liquid specimen collection container, the container further includes a transfer means positioned inside the recessed portion of the lid. The transfer means is in liquid communication with the flexible extraction tube and used in transferring liquid from the cup to another vessel.

In another embodiment of the specimen collection container, the base of the cup has a tapered or slanted shape and the lowest gravitational point of the base is at its center. In variations of this embodiment, the tapered or slanted base has an added depression at its lowest gravitational point.

In yet another embodiment of the specimen collection container of the present invention, the cup further includes a locking means selected from a group of locking means consisting of a plurality of threads or one or more flanges. In still another embodiment of the specimen container, the base of the cup includes more than one depression. The depressions are distributed substantially circularly on the base. Each of these depressions has the same depth, that depth being the lowest gravitational point on the base. When the lid is in locked engagement with the locking means, one of the depressions is aligned with the recessed portion and the flexible extraction tube extends into and reaches the bottom surface of that depression. In another embodiment of the liquid specimen collection container, the base of the cup further contains a channel connecting the plurality of depressions.

In another embodiment of the specimen collection container according to the present invention, the flexible extraction tube extends into and reaches the bottom surface of one of the depressions, the depression lying adjacent to the sidewall of the cup and the flexible extraction tube resting against and being partially supported by the sidewall.

In yet another embodiment of the specimen collection container, the flexible extraction tube has an accordion-like shape.

In yet another aspect of the present invention there is provided a system for drawing off liquid to be analyzed in a laboratory. The system includes a liquid specimen collection container and a vessel. The liquid specimen collection container includes a cup and a lid. The cup has a base, a sidewall extending substantially transversely from the base, terminating in an edge portion, such that the base and the sidewall form a cavity. The lid is formed for leak-resistant seating on the edge portion. The lid has a recessed portion having formed therein a generally hollow protrusion terminating in a free end which, when the lid sits on the edge portion of the cup, extends into the interior of the cup. The free end is spaced apart from the base. A flexible extraction tube is present, the tube having a first end affixed to the free end of the protrusion and a second end arranged to lie on the base of the cup. The second end of the flexible extraction tube is immersed in a liquid disposed in the cup. The cup also includes a transfer means positioned inside the recessed portion of the lid and in liquid communication with the flexible extraction tube. The vessel is arranged for receiving a sample of liquid drawn from the specimen container. The vessel is in liquid communication with the container by the transfer means and the flexible extraction tube. In the presence of a suction force applied to the transfer means, the transfer means and the flexible extraction tube transfer the suction force to the liquid so as to cause liquid to be drawn into the tube, through the transfer means and into the vessel.

In an embodiment of the system, the vessel is an evacuated test tube having a closed bottom, an open top, cylindrical sidewalls extending therebetween, and a penetrable membrane attached to the open top. The penetrable membrane is penetrable by the transfer means such that the pressure differential between the container and the test tube allows for liquid to be drawn into the tube via the transfer means and flexible extraction tube.

In an embodiment of the system, the base of the cup has a tapered or slanted shape where the lowest gravitational point is at the center of the base.

In another embodiment of the system, the cup further includes a locking means selected from a group of locking means consisting of a plurality of threads or at least one flange. The base of the cup includes a plurality of depressions distributed substantially circularly on the base, each of the depressions having the same depth, that depth being the lowest gravitational point on the base. When the lid is in locked engagement with the locking means, the recessed portion is aligned with one of the depressions and the flexible extraction tube extends into and reaches the bottom surface of that depression. Sometimes, the flexible extraction tube extends into and reaches the bottom surface of one of the depressions, and the depression lies adjacent to the sidewall of the cup with the tube resting against, and being partially supported by, the sidewall.

In yet another embodiment of the system, the cup further includes a locking means selected from a group of locking means consisting of a plurality of threads or at least one flange. The base of the cup includes a plurality of depressions distributed substantially circularly on the base and the base contains a channel connecting the plurality of depressions. Each of the depressions has the same depth, that depth being the lowest gravitational point on the base. When the lid is in locked engagement with the locking means, the recessed portion is aligned with one of the depressions and the flexible extraction tube extends into and reaches the bottom surface of that depression. Sometimes, the flexible extraction tube extends into and reaches the bottom surface of one of the depressions and the depression lies adjacent to the sidewall of the cup with the tube resting against, and being partially supported by, the sidewall.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in greater detail than is necessary for a fundamental understanding of the invention, the description taken with the drawings making apparent to those skilled in the art how the several forms of the invention may be embodied in practice.

In the drawings:

FIG. 1A is a schematic side view of a specimen collection container constructed according to a first embodiment of the present invention;

FIG. 1B is a schematic top view of the container shown in FIG. 1A;

FIG. 1C is a schematic side view of a flexible extraction tube which may be used with the container appearing in FIG. 1A;

FIG. 2 is a schematic side view of a specimen collection container constructed according to a second embodiment of the present invention;

FIG. 3A is a schematic side view of a specimen collection container constructed according to a third embodiment of the present invention;

FIG. 3B is a schematic top view of the container shown in FIG. 3A;

FIG. 3C is a schematic side view of a flexible extraction tube which may be used with the container appearing in FIG. 3A;

FIG. 4A is a schematic side view of a specimen collection container constructed according to a fourth embodiment of the present invention;

FIG. 4B is a schematic view of the base of the cup of the container shown in FIG. 4A;

FIG. 5 is a schematic side view of a specimen collection container constructed according to a fifth embodiment of the present invention; and

FIG. 6 is a schematic side view of another embodiment of the flexible extraction tube used in specimen collection containers constructed according to the present invention.

Similar elements in the Figures are numbered with similar reference numerals.

### DETAILED DESCRIPTION OF PREFERRED EMBODIMENTS

Transferring bodily fluids for laboratory analysis, particularly urine, from a liquid specimen collection container used in collecting these fluids, is difficult in all cases. This is even truer when there is only a small volume of collected fluid. Typically, urinalysis requires samples of at least 5 ccs; multiples of this minimum volume are required when multiple analyses are to be analyzed.

Patients often find it difficult to provide the required minimum quantity of liquid on demand and samples of smaller than 5 ccs are often collected. In order to use such small samples, a specimen collection container is herein provided which includes a flexible extraction tube. The tube is constructed to reach the bottom of the container and is affixed to a protrusion which extends downward from the lid of the specimen collection container. Such a configuration is absent in prior art where only rigid plastic tubes are used to draw off samples of the collected fluid. Additionally, prior art rigid tubes have lengths that are typically at least 3 mms short of the bottom of the container.

A flexible extraction tube reaching the bottom of the cup obviates the need for tilting the container when extracting small samples for analysis. Additionally, pressing down on a test tube positioned in a recess of the lid in order to have a rigid plastic tube more closely approach the bottom surface of the container is unnecessary when using the present invention. This prevents damage to the container and its parts and inadvertent injury to a technician from sharp, often biologically contaminated, objects such as needle cannulas or test tubes. It is also envisioned that the specimen collection container constructed according to embodiments of the present invention will allow use of substantially all of the collected fluid, even when the volume of liquid is small.

In what is discussed and described herein reference is made to urine. However, it is to be understood that the specimen collection containers described herein may also be used in collecting other bodily fluids.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

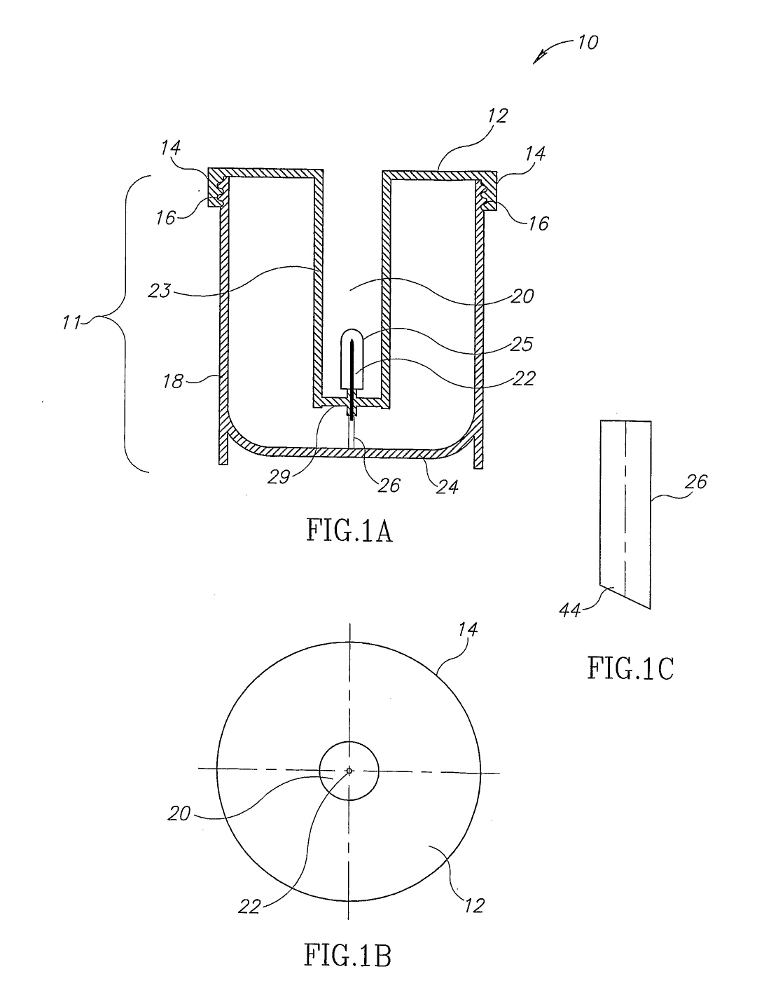

Reference is now made to FIG. 1A which shows a side view of a liquid specimen collection container 10, constructed and operative in accordance with a first embodiment of the present invention. Container 10 includes a cup 11 and a lid 12. Cup 11 has a generally cylindrical shape and lid 12 is configured to include a cylindrical recess 20. Cup 11 includes sidewalls 18 extending from base 24 to an open top (not visible), the open top here being covered by lid 12. Lid 12 may be joined to sidewalls 18 by threads 16 on lid edge 14. In other embodiments, lid 12 may be joined to the sidewalls 18 of cup 11 by at least one flange-like element extending from the sidewalls 18 of the cup allowing for a snap-on locking engagement. As is readily evident, cup 11 and lid 12 may be made from any of many plastic materials known to those skilled in the art.

FIG. 1A shows lid 12 as having only a single cylindrical recess 20. In other embodiments, more than one recess may be present in lid 12. As shown in FIG. 1B, the cylindrical recess 20 in the embodiment shown in FIG. 1A is positioned at the center of lid 12. In other embodiments, as will be discussed below, the one or more cylindrical recesses may be positioned at a distance removed from the center of lid 12. Additionally, in yet another embodiment, recess 20 may be configured to have a shape other than cylindrical.

Recess 20 generates a protrusion 23 extending downward into cup 11 in the direction of base 24. Extending from protrusion 23 is a flexible extraction tube 26 which extends to the base 24 of cup 11. The increased length of extraction tube 26 allows for substantially greater amounts of liquid to be withdrawn from container 10 even when the container sits level on an external surface. Additional pressure is not required to be placed on an evacuated receiving test tube to bring the flexible extraction tube 26 into closer proximity to base 24. Additionally, because of the flexibility of tube 26, if additional pressure would be exerted, damage to container 10 would not occur. Without limiting the materials that may be used in fabricating flexible extraction tube 26, tube 26 may be made from silicones, polyurethane (PU) rubber or poly vinyl chloride (PVC).

Flexible extraction tube 26 is sealed to the bottom face 29 of protrusion 23. Sealing may be effected by hot welding or solvent welding, two techniques both well-known to those skilled in the art of working with polymeric materials. Alternatively, flexible tube 26 may be mechanically joined to protrusion 23 by forcing tube 26 to fit over a fitted portion of protrusion 23. The above techniques are exemplary methods for joining tube 26 to protrusion 23 and are not deemed to be an exhaustive list.

Tube 26 is in liquid communication with needle cannula 22 which extends from the bottom of recess 20 in an upward direction toward lid 12 and away from base 24 of cup 11. Needle cannula 22 is used to pierce an evacuated test tube (not shown) when a sample must be drawn off from container 10. When not in use, needle cannula 22 is covered by protective needle case 25, which typically, but without being limiting, is made of silicone rubber. When the evacuated test tube is positioned in recess 20 and pressed down, protective needle case 25 also moves downward exposing needle cannula 22. The needle is then free to puncture a penetrable membrane covering the mouth of the test tube. After the test tube is filled, the tube is removed, its membrane reseals, and protective needle case 25 moves upward again covering the needle preventing unintentional injuries. The needle cannula 22 is also at times referred to herein as a transfer means.

FIG. 1B, to which reference is now made, shows a top view of specimen collection container 10 with recess 20 centrally positioned in lid 12. It should be readily understood, and as will be presented in other embodiments below, recess 20 need not be centrally located but may be positioned away from the center of lid 12.

The end of flexible extraction tube 26 closest to the base 24 of cup 11 may have a slanted edge 44 as is shown in FIG. 1C now referred to. This slanted configuration however is not intended to limit other possible configurations of tube 26.

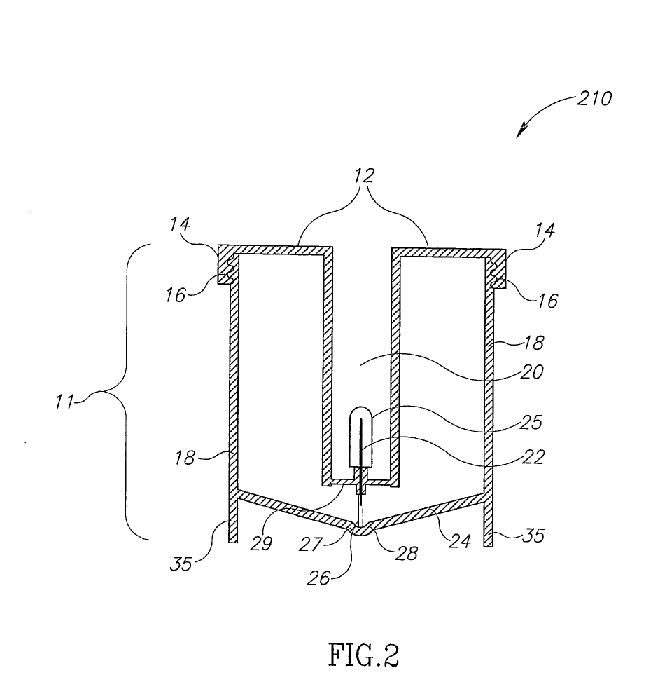

Reference is now made to FIG. 2 where a specimen collection container constructed according to a second embodiment of the present invention is shown. Specimen collection container 210 includes all the elements of specimen collection container 10 discussed above in conjunction with FIG. 1A. These elements are similarly numbered. Additionally, in this second embodiment, the base 24 of cup 11 is slanted or tapered, allowing small amounts of collected liquid to concentrate at tapered end 27. Tapered end 27 may also include an additional depression 28 which allows for gathering of an even smaller amount of fluid. Flexible extraction tube 26 is positioned so as to reach the bottommost portion of slanted base 24, and when depression 28 is present, to reach the bottom of depression 28. Since base 24 is not flat, the sidewalls 18 of cup 11 may extend downward circumferentially forming sidewall extensions 35 allowing for a level stable positioning of container 210 on an external surface.

A ring (not shown) additionally and optionally may be affixed to sidewalls 18 of cup 11. This ring would act to further stabilize container 210 allowing it to remain level and stable when placed on a surface.

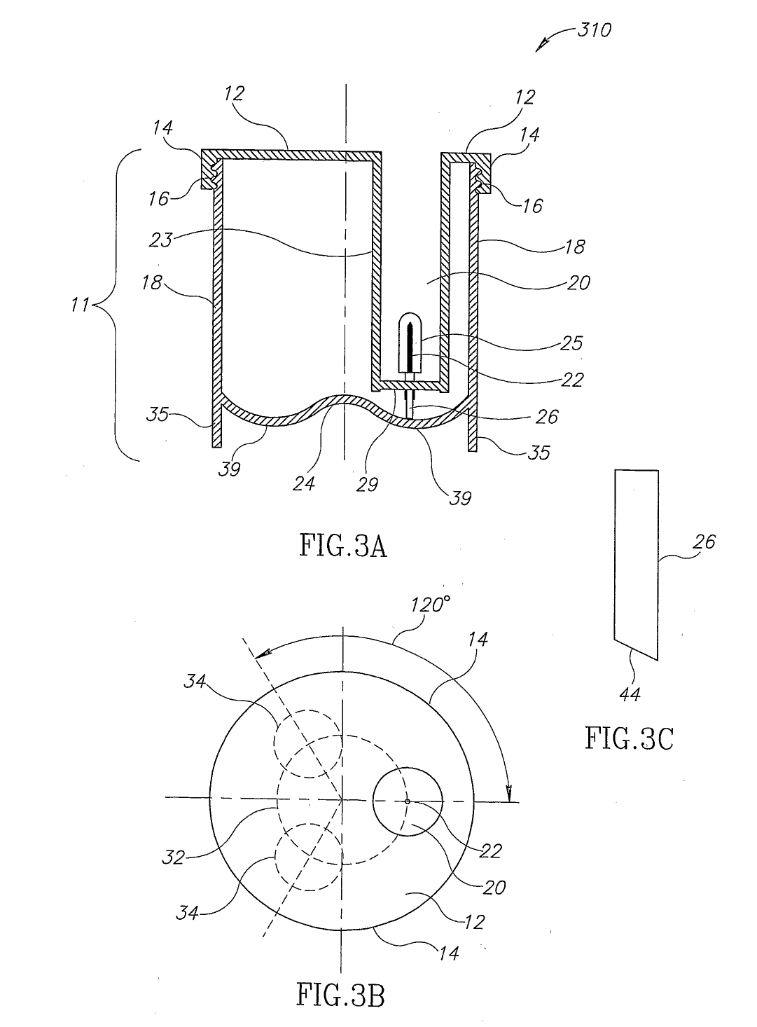

Reference is now made to FIG. 3A where a specimen collection container constructed according to a third embodiment of the present invention is shown. Specimen collection container 310 includes all the elements of the specimen collection container in FIG. 1A and these elements are similarly numbered. Additionally, base 24 of cup 11 of container 310 contains several depressions 39 into which urine may collect from base 24.

In the embodiment of FIG. 3A, recess 20 is positioned off-center of lid 12 and flexible extraction tube 26 extends into the bottom of one of the depressions 39. As shown by the broken lines in FIG. 3B, a schematic top view of the container in FIG. 3A, recess 20 may be positioned in any of three positions, each position approximately 120° apart from its neighbors. Dashed circles 34 represent the other two possible positions of recess 20 as lid 12 is rotated. Dashed line 32 indicates the path of travel of needle cannula 22 when moving between the three locked positions. These positions correspond to the standard three thread lock lids commercially available, which, with only a partial turn of the lid, allows the lid to tightly lock and engage with the cup. In these three positions, flexible tube 26 always extends into one of the three aligned depressions 39 positioned on base 24 of cup 11.

In FIG. 3C, now referred to, flexible tube 26 may have a slanted edge 44 at its end closest to base 24 of cup 11 of container 310. This slanted configuration, however, is not intended to limit other possible configurations of tube 26.

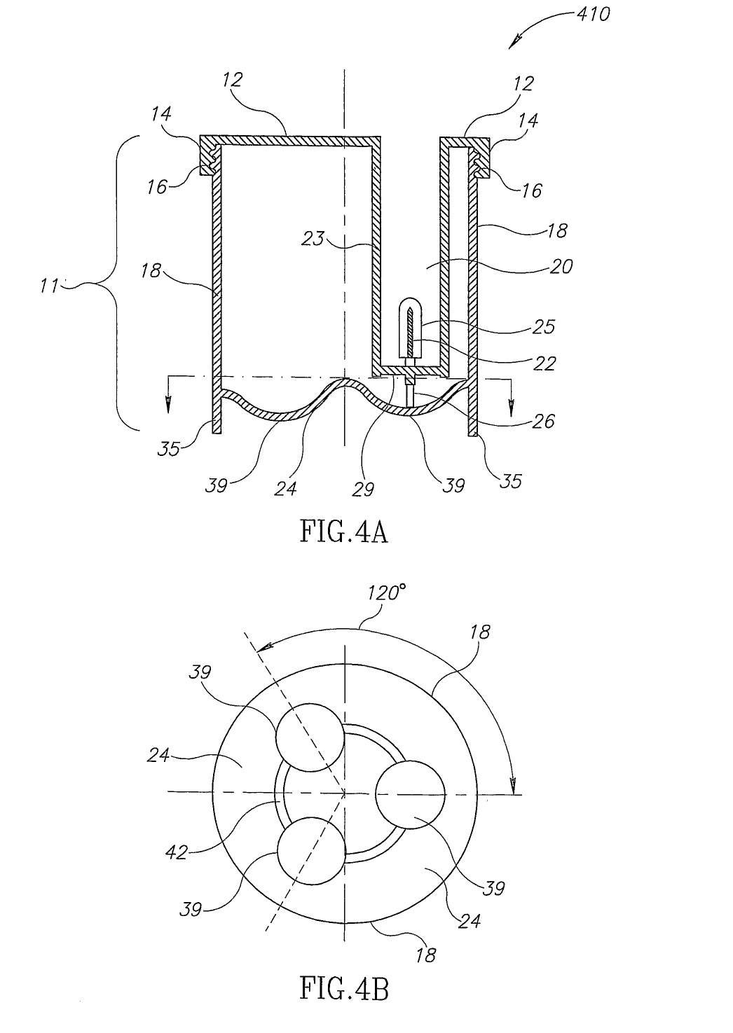

Reference is now made to FIG. 4A where a fourth embodiment of the present invention is shown. Specimen collection container 410 includes all the elements of the container in FIG. 1A and those of the container in FIG. 3A and these are similarly numbered. Additionally, base 24 of cup 11 of container 410 contains a symmetrical circumferential channel 42 (FIG. 4B) connecting the several depressions 39 located in base 24 of cup 11. Once again each of these depressions is aligned with one of the three thread lock positions of lid 12 and recess 20. Liquid gathers in this channel and then flows into depressions 39.

In this embodiment, recess 20 is positioned off center of lid 12 and flexible extraction tube 26 extends into the bottom of one of depressions 39 aligned with recess 20 when lid 12 is in one of its locked positions. As shown in FIG. 4B, a schematic view of base 24 of cup 11 in FIG. 4A, each of the three depressions 39 are positioned 1200 apart from its nearest neighbors. As in FIG. 3B, these positions correspond to and are aligned with one of the locked positions of conventional three thread lock lids commercially available. Only a partial turn of lid 12 is required to tightly lock it with cup 11. In these three positions, flexible extraction tube 26 always extends and reaches the bottom of one of the aligned depressions 39.

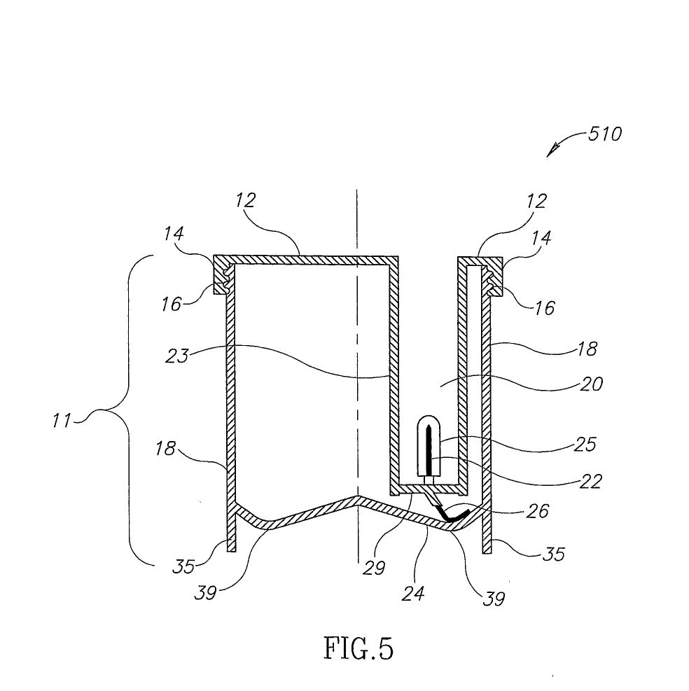

A specimen collection container 510 constructed in accordance with a fifth embodiment of the present invention is shown in FIG. 5. Specimen collection container 510 is similar to the one shown in FIG. 3A or FIG. 4A and identical elements are similarly numbered. Again recess 20 is off-center with respect to the center of lid 12. Flexible tube 26 lies close to sidewall 18 of cup 11 of container 510 allowing it to more easily draw off specimen gathered in depression 39. Tube 26 may even partially rest on sidewall 18 as shown in FIG. 5. Depression 39 may be, and usually is, also positioned very close to sidewall 18 in this embodiment.

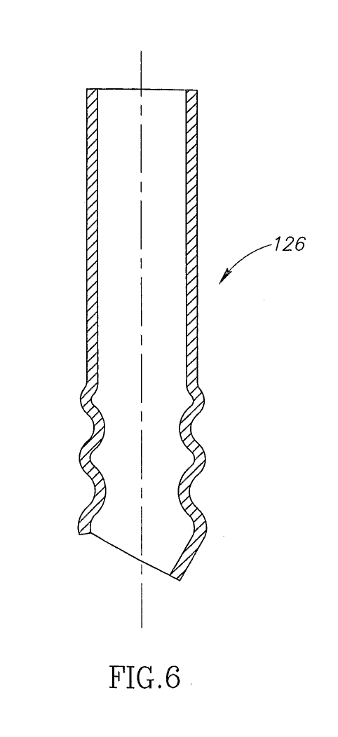

FIG. 6, now referenced, illustrates another embodiment of the flexible extraction tube. Tube 126 is constructed slightly differently from the tubes 26 shown in FIGS. 1A-5. Tube 126 is formed as an accordion-like tube, similar in shape to the shape of the bend in a soda straw. This shape allows for easier access to small volumes of fluid. Tube 126 may be fabricated from the same plastic materials used to produce the flexible extraction tubes 26 shown in FIGS. 1A-5.

In the above embodiments, samples have been described as being drawn off by an evacuated test tube with liquid passing to the test tube via flexible extraction tube 26 or 126 in liquid communication with needle cannula 22. The evacuated test tube is sized to be insertable into recess 20 during the drawing off process.

In the embodiments discussed previously, the flexible extraction tube is described as reaching the bottom surface of the container. In other embodiments the flexible tube may be long enough to lie flat along the bottom surface and may even curl back upon itself, possibly curling back even more than once.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

It will be appreciated by persons skilled in the art that the present invention is not limited by what has been particularly shown and described herein above. Rather the scope of the invention is defined by the claims that follow.

1. A liquid specimen collection container comprising:
a cup having a base, a sidewall extending substantially transversely from said base, terminating in an edge portion, such that said base and said sidewall form therein a cavity;
a lid formed for leak-resistant seating on said edge portion, and having a recessed portion having formed therein a generally hollow protrusion terminating in a free end which, when said lid is seated on said edge portion of said cup, extends into the interior of said cup such that said free end is spaced from said base; and
a flexible extraction tube having a first end affixed to said free end of said protrusion and a second end arranged to lie on said base, such that said second end is immersed in a liquid disposed in said cup, wherein, in the presence of a suction force applied to said recessed portion of said lid, said hollow protrusion and said flexible extraction tube are operative to transfer the suction force to the liquid so as to cause liquid to be drawn into said tube, through said protrusion and out of said collection container.

2. A liquid specimen collection container according to claim 1, further including a transfer means positioned inside said recessed portion of said lid, said transfer means in liquid communication with said flexible extraction tube and used in transferring liquid from said cup to another vessel.

3. A liquid specimen collection container according to claim 2, wherein said base of said cup has a tapered or slanted shape and where the lowest gravitational point is at the center of said base.

4. A liquid specimen collection container according to claim 3, wherein said tapered or slanted base has a depression at its lowest gravitational point.

5. A liquid specimen collection container according to claim 2, wherein said cup further includes a locking means selected from a group of locking means consisting of a plurality of threads or at least one flange.

6. A liquid specimen collection container according to claim 5, wherein said base of said cup includes a plurality of depressions distributed substantially circularly on said base, each of said depressions having the same depth, that depth being the lowest gravitational point on said base, and when said lid is in locked engagement with said locking means, said recessed portion is aligned with one of said depressions and said flexible extraction tube extends into and reaches the bottom surface of that depression.

7. A liquid specimen collection container according to claim 6, wherein said base of said cup further contains a channel connecting said plurality of depressions.

8. A liquid specimen collection container according to claim 6, wherein said flexible extraction tube extends into and reaches the bottom surface of one of said depressions, said depression lying adjacent to said sidewall of said cup and said tube resting against and being partially supported by said sidewall.

9. A liquid specimen collection container according to claim 1, wherein said flexible extraction tube has an accordion-like shape.

10. A liquid specimen collection container according to claim 1, wherein said base of said cup has a tapered or slanted shape and where the lowest gravitational point is at the center of said base.

11. A liquid specimen collection container according to claim 10, wherein said tapered or slanted base has a depression at its lowest gravitational point.

12. A liquid specimen collection container according to claim 1, wherein said cup further includes a locking means selected from a group of locking means consisting of a plurality of threads or at least one flange.

13. A liquid specimen collection container according to claim 12, wherein said base of said cup includes a plurality of depressions distributed substantially circularly on said base, each of said depressions having the same depth, that depth being the lowest gravitational point on said base, and when said lid is in locked engagement with said locking means, said recessed portion is aligned with one of said depressions and said flexible extraction tube extends into and reaches the bottom surface of that depression.

14. A liquid specimen collection container according to claim 13, wherein said base of said cup further contains a channel connecting said plurality of depressions.

15. A liquid specimen collection container according to claim 13, wherein said flexible extraction tube extends into and reaches the bottom surface of one of said depressions, said depression lying adjacent to said sidewall of said cup and said tube resting against and being partially supported by said sidewall.

16. A system for drawing off liquid to be analyzed in a laboratory, said system including:
a liquid specimen collection container, said container comprised of:
a cup having a base, a sidewall extending substantially transversely from said base, terminating in an edge portion, such that said base and said sidewall form therein a cavity;
a lid formed for leak-resistant seating on said edge portion, and having a recessed portion having formed therein a generally hollow protrusion terminating in a free end which, when said lid is seated on said edge portion of said cup, extends into the interior of said cup such that said free end is spaced from said base;
a flexible extraction tube having a first end affixed to said free end of said protrusion and a second end arranged to lie on said base, such that said second end is immersed in a liquid disposed in said cup; and
a transfer means positioned inside said recessed portion of said lid and in liquid communication with said flexible extraction tube; and
an evacuated vessel for receiving a sample of liquid drawn from said specimen container, said vessel being in liquid communication with said container by said transfer means and said flexible extraction tube and wherein, in the presence of a suction force applied to said transfer means, said transfer means and said flexible extraction tube are operative to transfer the suction force to the liquid so as to cause liquid to be drawn into the tube, through the transfer means and into said vessel.

17. A system according to claim 16, wherein said vessel is an evacuated test tube having a closed bottom, an open top, cylindrical sidewalls extending therebetween, and a penetrable membrane attached to said open top, said penetrable membrane being penetrable by said transfer means such that the pressure differential between said container and said test tube allows liquid to be drawn into said tube via said transfer means and said flexible extraction tube.

18. A system according to claim 16, wherein said base of said cup has a tapered or slanted shape and where the lowest gravitational point is at the center of said base.

19. A system according to claim 16, wherein said cup further includes a locking means selected from a group of locking means consisting of a plurality of threads or at least one flange, and said base of said cup includes a plurality of depressions distributed substantially circularly on said base, each of said depressions having the same depth, that depth being the lowest gravitational point on said base, and when said lid is in locked engagement with said locking means, said recessed portion is aligned with one of said depressions and said flexible extraction tube extends into and reaches the bottom surface of that depression.

20. A system according to claim 19, wherein said flexible extraction tube extends into and reaches the bottom surface of one of said depressions, said depression lying adjacent to said sidewall of said cup and said tube resting against and being partially supported by said sidewall.

21. A system according to claim 16, wherein said cup further includes a locking means selected from a group of locking means consisting of a plurality of threads or at least one flange and said base of said cup includes a plurality of depressions distributed substantially circularly on said base and said base contains a channel connecting said plurality of depressions, each of said depressions having the same depth, that depth being the lowest gravitational point on said base, and when said lid is in locked engagement with said locking means, said recessed portion is aligned with one of said depressions and said flexible extraction tube extends into and reaches the bottom surface of that depression.

22. A system according to claim 21, wherein said flexible extraction tube extends into and reaches the bottom surface of one of said depressions, said depression lying adjacent to said sidewall of said cup and said tube resting against and being partially supported by said sidewall.
